(19) United States
(12) Patent Application Publication
(10) Pub. No.: US 2010/0094332 A1
(43) Pub. Date: Apr. 15, 2010

(54) Device and Method for Altering Cardiac Activity

(76) Inventor: Peter Willshaw, Swansea (GB)

Correspondence Address: WADDEY & PATTERSON, P.C., 1600 DIVISION STREET, SUITE 500, NASHVILLE, TN, 37203, US

(21) Appl. No.: 12/374,163

(22) PCT Filed: Jul. 18, 2007
(86) PCT No.: PCT/GB07/02716

§ 371 Date: Dec. 15, 2009

(30) Foreign Application Priority Data
Jul. 18, 2006 (GB) 0614218.6

(51) Int. Cl. A61B 17/00; A61B 5/0456; A61B 8/00

(52) U.S. Cl. 606/204; 600/509; 600/453

(57) ABSTRACT

A device for altering cardiac activity, said device comprising a neck engaging member, said neck engaging member having at least one pressure applicator provided as a predefined area which in use comes into contact with and occludes or partially occludes at least one carotid artery, said device including a control mechanism which is operable to cause the pressure applicator to rapidly occlude or partially occlude the artery in order to provoke heart rate turbulence.

The invention relates to a device and method for altering cardiac activity so that information obtained from monitoring the activity of the heart can be used in the prediction of cardiac events. In particular but not exclusively, the invention relates to a device and method for non-invasively provoking, when required, heart rate changes in individuals such as humans or animals by the occlusion or partial occlusion of one or more carotid arteries of that individual.

The heart comprises two thin-walled atrial chambers, which provide a left and a right atrial chamber and these chambers sit above the two thicker walled and larger, left and right ventricular chambers of the heart. The right ventricular chamber pumps blood to the lungs, while the left ventricular chamber pumps blood to the rest of the body. The atrial chambers pump blood to fill the two ventricular chambers before they contract to pump blood to the body. The heart has its own natural or built in pacemaker called the sinoatrial node (also called the SA node or sinus node). The SA node sends impulses to the right and left atrial chambers so they are caused to beat. Impulses are then sent via the atrioventricular (AV) node to the ventricular chambers causing them to beat a split second later.

The carotid arteries in the neck just below the angle of the jaw contain nerve endings within their walls, which are stretched by each blood pressure pulse. These nerves are called baroreceptors and with each pulse of pressure they send impulses to the brain centres involved in blood pressure control. High pressures produce more nerve impulses and low pressures produce fewer impulses. Under normal conditions, this nerve traffic controls the heart to produce the normal heart rate of around 70 beats per minute. If the pressure rises, the heart is controlled such that the heart rate falls. However, if the blood pressure falls as a result of a fall in the heart rate due to missed beats, the nerve traffic is inhibited and the heart speeds up. When the heart begins beating normally again following a ‘compensatory pause’ and the normal pattern of blood pressure pulses is restored, the heart rate slows down again and the rate may even fall below that present before the premature ventricular contraction (PVC). The temporal pattern of these heart rate changes is Heart Rate Turbulence (HRT).

Changes in the regular beat of the heart (arrhythmias) can occur and they tend to occur more commonly in individuals as they become older and in particular in middle age, although they may occur in younger individuals. In younger individuals, arrhythmias may be due to genetically inherited heart defects. Arrhythmias may be associated with and provide an indication of heart disease, which could lead to a heart attack (myocardial infarction). A heart attack occurs as the result of muscle cells in the heart dying and as result of the lack of supply of oxygen to the heart and nutrients. Heart attacks may be due to poor health due to the blocking of arteries or poor circulation but there may also be genetic defects in the individual, which cause heart muscle to be damaged. If the heart disease goes undetected the individual has an increased risk of death.

Abnormalities in the heart rhythm have been controlled by a range of methods, including prescribing drugs to control the heart rate, using devices such as automatic pacemakers and/or defibrillators, which are implanted in the patient or alternatively surgery can be used. These techniques are usually used when the heart disease is more advanced as treatment is usually sought after an event such as a heart attack, which demonstrates that there is already damage to the heart tissue. An example of a device that is inserted in the body to control heart rate is discussed in U.S. Pat. No. 5,222,980, which describes an implantable heart assist device including an extra-aortic balloon pump which uses stimulation of nearby muscles to assist heart activity. However, there is patient trauma when inserting such devices in the body and the patient, who has to undergo an operation so that the device can be inserted. When undergoing such operations, patients need to be anaesthetized, which may have particular risks for patients with heart problems.

It has become apparent that predicting whether an individual is likely to have a cardiac event is a preferable way to dealing with heart disease by treating a person once the heart disease has progressed to a more serious stage and where there may be more pronounced damage to the heart. If treatment can be given as early as possible, then this has the benefit of maintaining the health of the individual and also avoids costs by reducing the reliance on costly forms of treatment such as surgery.

Investigations have been made into how the heart can be monitored to see if there is a likelihood of a cardiac event such as a heart attack occurring.

Schmidt et al (in Heart Rhythm 2004 pp 732-738, 1999) were the first to describe Heart Rate Turbulence (HRT) as a way of predicting Heart Disease. HRT refers to the changes in heart rate following a premature ventricular contraction (PVC) of the left ventricle. The changes in heart rate are characterised by two parameters, turbulence onset (TO) and turbulence slope (TS). These parameters were shown to be powerful predictors of subsequent cardiac events in patients who had suffered a myocardial infarction. PVCs occur spontaneously even in healthy individuals, who often refer to them using the expression ‘my heart missed a beat’.

To date, measuring premature ventricular contractions necessary for the calculation of HRT parameters are almost universally obtained from 24 hour recordings using an Electrocardiogram (ECG), where the patient is fitted with a small highly portable ECG recorder known as a Holter. With luck, the patient will display sufficient PVCs to allow HRT to be calculated. However, it has been shown that in this 24 hour time frame, not all people will show PVCs and so individuals that may be at risk from heart disease could go undetected. An example of this is that in studies it has been found that in a sample of 110 healthy volunteers only 43 showed PVCs in their 24 Hr Holter recording i.e. 39%, so illustrating that many individuals with potential heart disease are not diagnosed. Also, there is the disadvantage that because monitoring occurs over a relatively long period that individuals may not want to use the monitoring device because it could impinge on their lifestyle, for example if they want to indulge in activities such as swimming.

To overcome the disadvantages of long periods of monitoring it has been suggested that HRT in patients is studied in patients when fitted with pacemakers or with programmable defibrillators. However, this has the disadvantage that monitoring is only being carried out for individuals that warrant the need for a defibrillator, i.e. their heart disease may well be advanced because health authorities are unlikely to go to the expense of implanting equipment where it is not vital to do so. Also there is an increased risk to the patient in that they have to undergo surgery for the device to be implanted.

Given the clinical importance of HRT as a predictor of subsequent cardiac events but the difficulty of obtaining sufficient PVCs from most patients, the present invention seeks to address the identified need for developing a device or method which is capable of provoking at will the same heart rate changes as those observed following a premature ventricular contraction. The essence of such a device would be to prevent or attenuate the carotid baroreceptor response to one or more pressure pulses i.e. to make the baroreceptors ‘miss a beat’ or ‘miss’ more than one beat.

The idea of loading and unloading the carotid baroreceptors by applying neck suction and neck pressure respectively is not new.

Devices that have been used until now have employed compression and suction of the neck tissues using a transmission fluid between a chamber containing the fluid and the neck tissues. The known devices involve compressing the neck tissue for a number of seconds in order to evoke a steady-state response but there is no compression that is synchronised with the R-wave associated with cardiac activity. The R wave is shown as a spike on an ECG print out and indicates the point at which the heart ventricles are about to eject blood.

Devices have been developed where a moulded neck chamber is connected to a bellows system and is placed around the neck. The bellows generates positive pressure on the neck and makes a beat-by-beat transition to negative pressure over about 10 beats in a step-like fashion with each step transition being triggered by an R-wave measured from cardiac activity. The objective of this device was to generate a baroreceptor sensitivity curve over a wide range of pressures. However, this device does not provoke HRT because of the slow delivery of pressure and it is not capable of producing very fast changes of neck pressure to provoke a response that could be used to measure the risk of cardiac disease.

The current invention seeks to overcome the problems associated with the prior art by providing a device and method that can provoke heart rate changes at will without harming the individual.

According to the present invention there is provided a device for altering cardiac activity, said device comprising a neck engaging member, said neck engaging member having at least one pressure applicator provided as a predefined area which in use comes into contact with and occludes or partially occludes at least one carotid artery, said device including a control mechanism which is operable to cause the pressure applicator to rapidly occlude or partially occlude the at least one carotid artery in order to provoke heart rate turbulence.

It is preferred that the device includes a control mechanism that causes the pressure applicator to occlude or partially occlude the at least one carotid artery and release therefore after a predetermined period of time.

It is envisaged that the occlusion or partial occlusion of the at least one carotid artery occurs is achieved within a period of a few milliseconds following the command to occlude or partially occlude. The time periods are typically 2, 3, 4, 5 or 10 or 20 or more milliseconds and that the time period during which the occlusion or partial occlusion is maintained is of one or more cardiac cycles in duration.

Preferably the neck engaging member device comprises a cuff that is placed around the neck of the individual, with the pressure applicator being aligned with the at least one carotid artery. The pressure applicator may comprise an inflatable balloon. The balloon can be inflated with a liquid or gas once in position with a carotid artery. Alternatively, the pressure applicator is a mechanical foot that is brought into contact with and presses against the one or more carotid arteries to fully or partially occlude them.

In a further arrangement, the neck engaging member comprises one or more arms that come into contact with the one or more carotid arteries.

In a preferred arrangement, the neck engaging member includes a sensor, which detects the carotid pulse when the pressure applicator is positioned over the carotid artery. By having locating means, such as a sensor, this allows health care professionals to easily position the neck engaging member such as the cuff or arms in the correct location for the carotid artery to be occluded or partially occluded.

In an alternative arrangement, visible indicators are present so that the neck engaging member can be aligned with the at least one carotid artery.

It is envisaged that the pressure applicator is a mechanical foot that is brought into contact with the at least one carotid artery.

Preferably, the control mechanism is associated with a cardiac monitoring sensor to detect the R-wave associated with cardiac activity. The cardiac monitoring sensor is used to measure the time delay between the R-wave and ventricular ejection into the arterial system. This is of the order of substantially 50 milliseconds. Pressure must be applied as soon as possible following the R-wave to prevent the arterial pulse from arriving at the carotid sinus or to attenuate the magnitude of such pulse at the carotid sinus.

It is envisaged that the cardiac monitoring sensor is in communication with an actuator that is operable to cause the pressure applicator to come into contact with the at least one carotid artery following detection of the R-wave.

In a preferred arrangement, the activation of the actuator is triggered by detection of the R-wave followed by which the actuator is caused to release so that zero pressure is applied to the at least one carotid artery.

It is preferred that the actuator is a voice coil actuator. As previously mentioned, the pressure applicator comes into contact with the at least one carotid artery within a few milliseconds following the detected R-wave.

In a preferred arrangement, the device includes a pressure measuring device that monitors the pressure of the pressure inducing device against the carotid artery.

The pressure measuring device is provided as a manometer.

It is envisaged that the pressure measuring device provides a pressure feedback signal as controller to a control system for the sensor for the pressure applicator which is compared with a desired pressure reference signal. The use of a reference signal for the pressure applied is a safety feature which allows the force developed by an actuator for the pressure applicator to be expressed in terms of pressure in a controlled fashion.

In one embodiment, the reference signal is limited.

In another embodiment, the current to the voice coil actuator is limited.

It is envisaged that both of these factors could be used to limit the pressure applied by the device.

It is preferred that the pressure applicator is associated with an ultrasound doppler probe resting on the neck to measure flow changes in the artery accompanying the pressure manoeuvre. The Doppler probe has a particular benefit in that it can measure for the presence of arterial plaques. In individuals with circulatory diseases, plaques may be dislodged from the walls of arteries and if this occurs, there is the risk of the plaque lodging in part of the circulatory system and causing blockages which if they occurred in organs such as the body or heart could kill an individual. The use of the Doppler probe would detect changes in the arterial wall structure and if there was a risk that the plaque is about to be dislodged, then use of the device could be halted.

It is envisaged that the device includes a pressure limiter. The use of a device that limits the pressure applied to the neck avoid the carotid artery being compressed too strongly or for too long a time period, which could disrupt blood flow in the individual.

Although the invention has been described with reference to the occlusion or partial occlusion of one carotid artery, both the left and right carotid arteries could be occluded or partially occluded. It is envisaged that the arteries are occluded or partially occluded substantially simultaneously but they may also be occluded or partially occluded sequentially.

Further, the invention has applications not only in monitoring the heart condition of humans, but also it can be used in the monitoring of animals. In particular, the invention has applications in the monitoring of valuable breeding stock such as horses, dogs or cattle where it is undesirable that genetic heart complaints are passed on.

According to a further embodiment of the invention, there is provided a method of occluding or partially occluding the carotid artery, said method comprising applying a neck engaging member to an individual's neck such that at least one pressure applicator provided as a predefined area comes into contact with at least one carotid artery, operating a control mechanism to cause the pressure applicator to rapidly occlude or partially occlude the at least one carotid artery in order to provoke heart rate turbulence.

Preferably the control mechanism is caused to occlude or partially occlude the at least one carotid artery and release therefore after a predetermined period of time.

It is envisaged that in a preferred arrangement, the device is caused to occlude or partially occlude the at least one carotid artery within milliseconds, typically 1 or more milliseconds and more typically within 5 to 10, 15 to 20 or more milliseconds.

An embodiment of the invention will now be described by way of example only with reference to the accompanying figures in which:

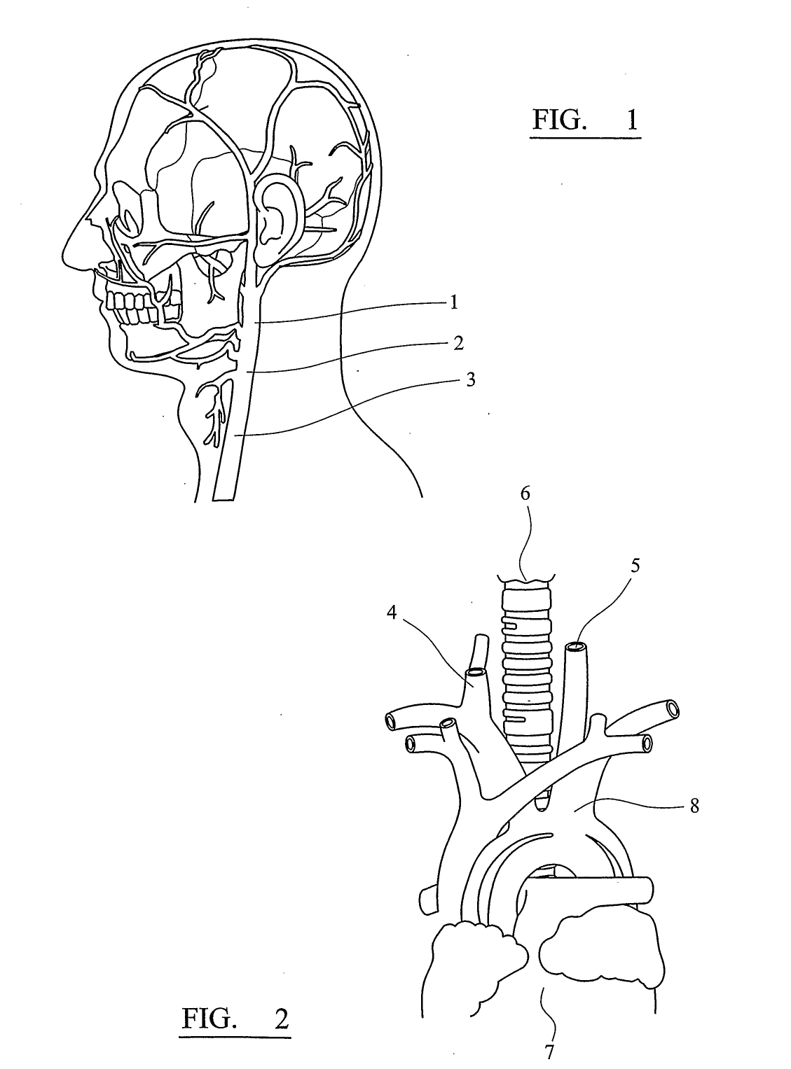

FIG. 1: shows the position of the carotid arteries for a human;

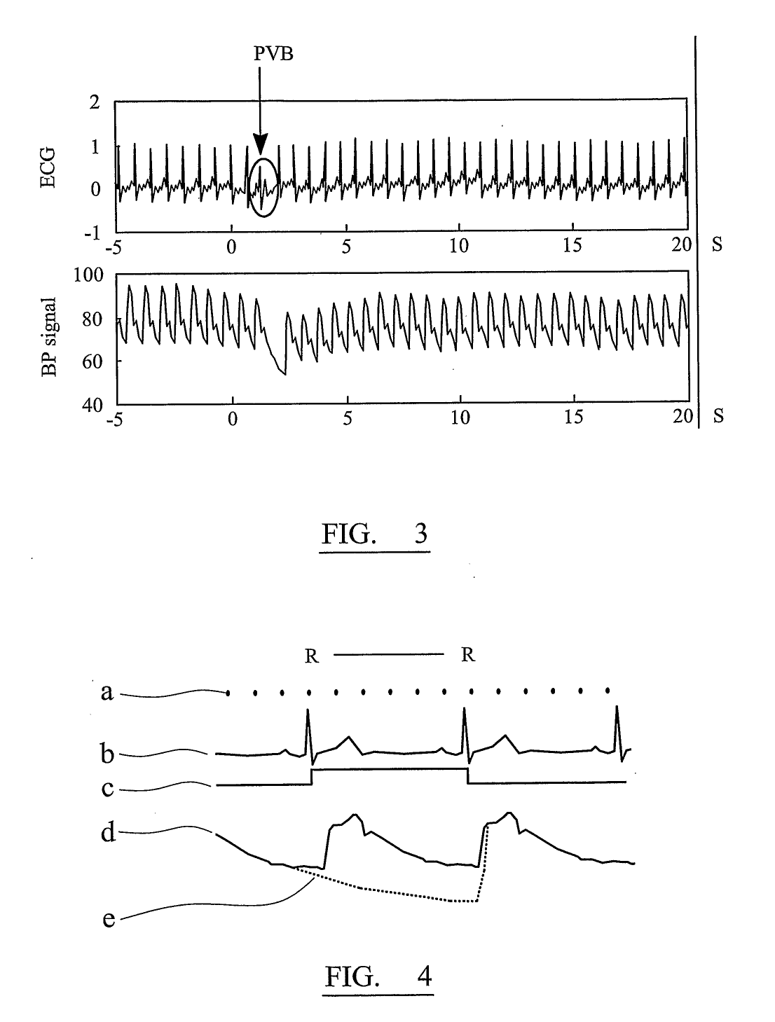

FIG. 2: shows a dissected view of the human heart with the position of the carotid arteries and baroceptors in the aortic arch;

FIG. 3: shows a pair of traces with the upper trace showing the electrical activity of the heart over time via using an ECG reading. The lower trace shows blood pressure over time;

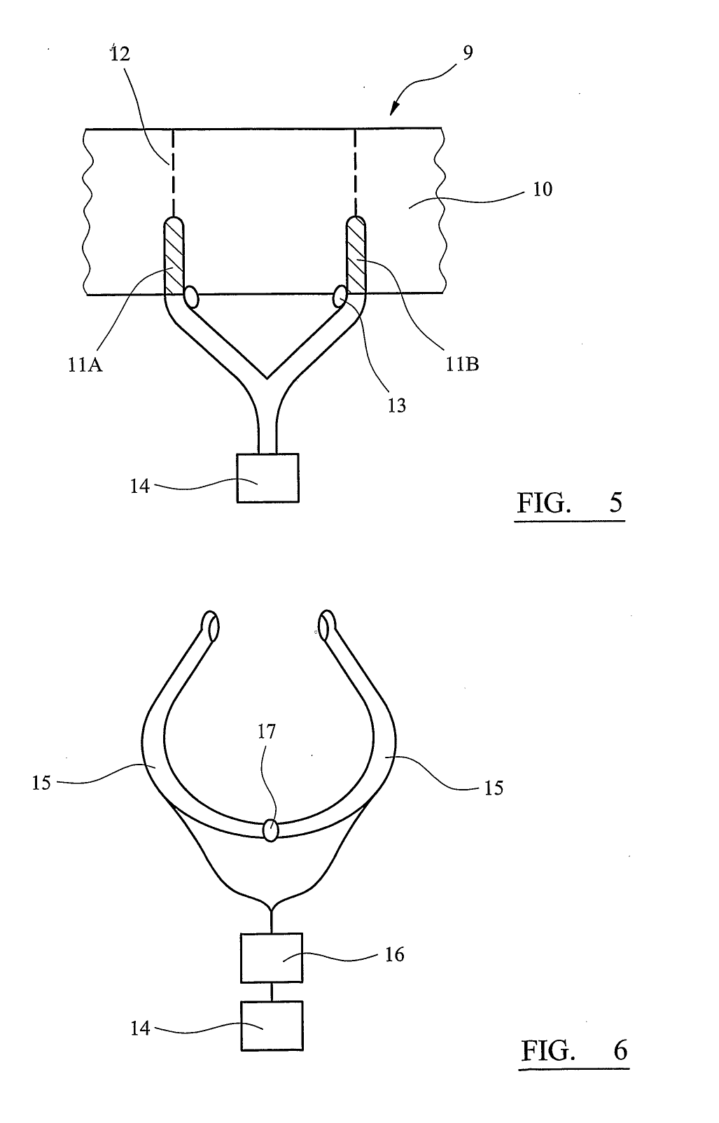

FIG. 4: shows the effect of applying pressure and not applying pressure to the carotid artery over time;

FIG. 5: shows a device for altering cardiac activity according to a first embodiment of the invention;

FIG. 6: shows a device for altering cardiac activity according to a second embodiment of the invention;

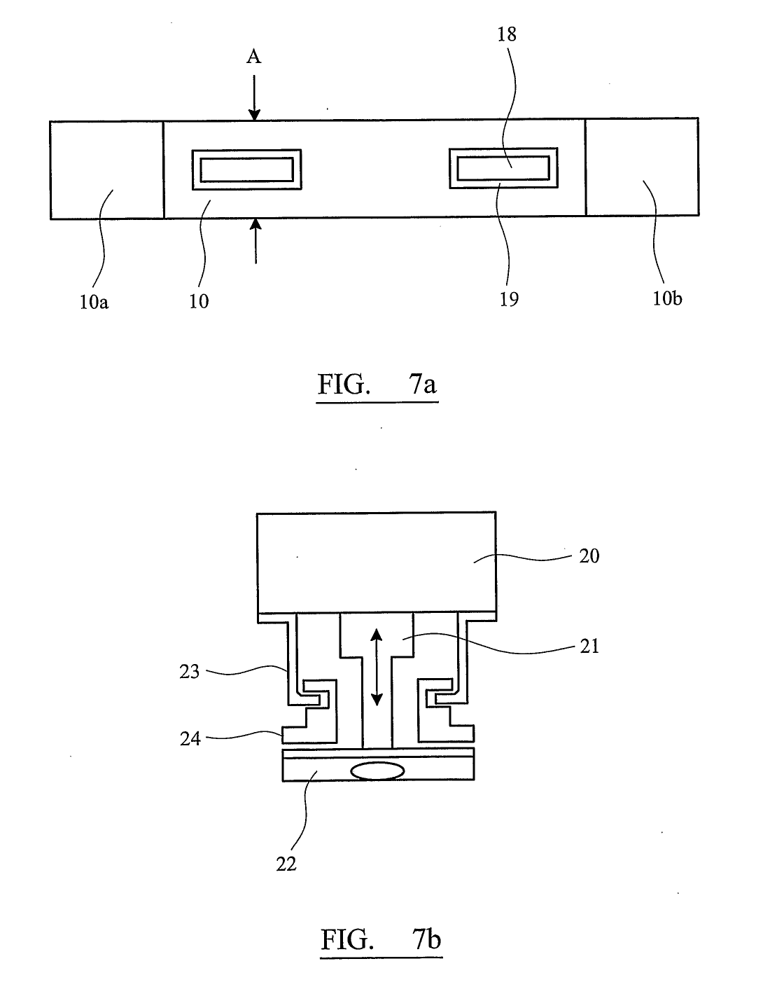

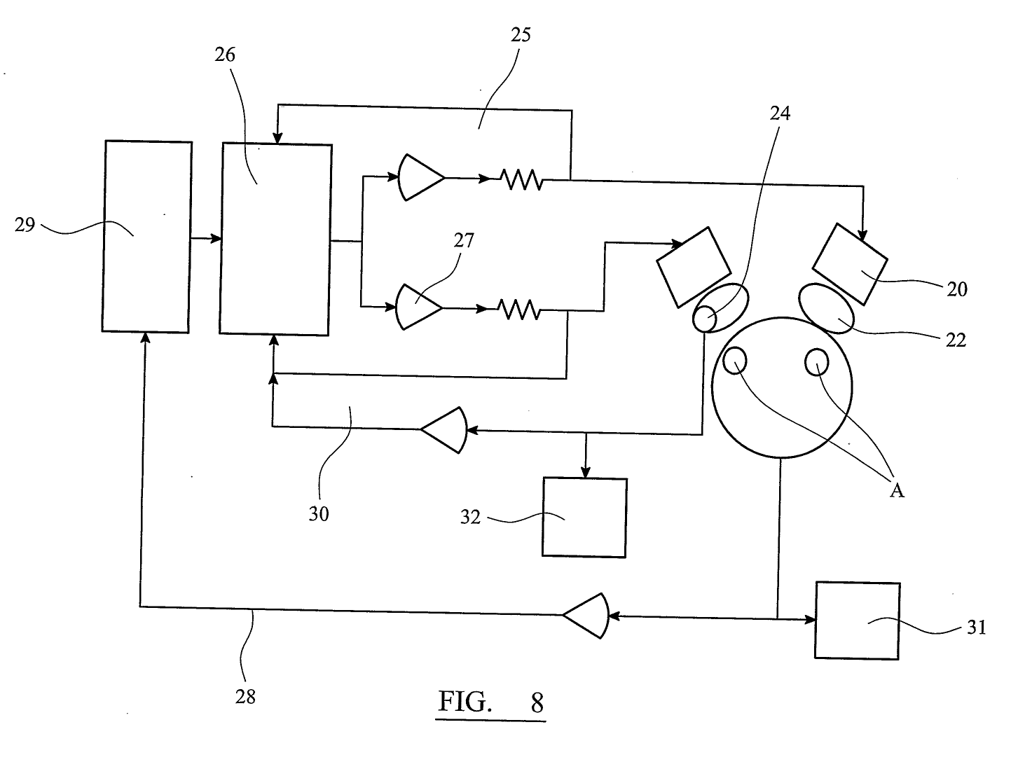

FIG. 7a: shows a schematic view of a further embodiment of the invention showing a device for altering cardiac activity;

FIG. 7b: shows a cross sectional view along A-A of FIG. 7a; and

FIG. 8: shows a schematic view of a device according to the invention with safety and control features for operation of the device.

As shown in FIG. 1, the carotid arteries are situated in the neck of an individual. The figure shows one side of the neck with the external carotid artery being shown as 1, the internal carotid artery is shown a 2, while the common carotid artery is shown as 3. There are left and right carotid arteries as shown in FIG. 2. The right common carotid artery is shown as 4, while the left carotid artery is shown as 5. The trachea is shown as 6, while the heart is shown at 7. On the aortic arch, there is a baroreceptor area 8.

The device of the present invention is designed to increase neck pressure in a controlled way for a single cardiac cycle thus unloading the baroreceptors for one beat and so simulating the pressure changes associated with a PVC to provoke HRT.

As shown in FIG. 3, the electrical activity of the heart (the ECG) is measured. The sharp upward spikes are known as R-waves and immediately precede ventricular contraction, which then normally causes ejection of blood from the heart into the arterial system. As shown in the figure a premature contraction has occurred and is labelled PVB. The lower trace shows the arterial pressure. The timescale is in seconds. Premature beats shown occur without producing ejection of blood from the ventricle and so blood pressure continues to fall before the next ejective beat begins the process of restoration of the normal pressure profile. This has the effect of the individual feeling that their heart has “missed a beat”.

FIG. 4 shows an electrocardiogram over time with the top line showing time in 100 millisecond intervals (a). The second line is the electrocardiogram trace over that time (b). The third line shows the intervals at which pressure is applied to the carotid artery over time (c). The bottom line shows the carotid pulse in the absence of pressure application (solid line—d) and the expected carotid pressure trajectory in the presence of applied pressure (dotted line—e). The values for HRT are not expressed as changes in heart rate but are expressed as the inverse of rate i.e. beat-to-beat interval known as R-R interval. The R-R intervals immediately precede ventricular contraction and ejection of blood from the heart. When the R-R intervals are plotted against time, before and after the PVC, heart rate turbulence is clearly evident by calculation of the turbulence onset (TO) and turbulence slope (TS). Turbulence onset quantifies shortening of RR interval, while Turbulence Slope is the greatest of slopes fitted to consecutive RR intervals after the VPC. Therefore the generation of a VPC is important to calculate these values.

As shown in FIG. 5 a device which can be used to provoke PVC according to the present invention is generally shown as 9 in FIG. 5, the device comprises a neck engaging member such as a cuff 10 which can encircle the neck. Preferably, the cuff is made of material that can be washed and also which has a degree of flexibility to accommodate different neck sizes. The cuff can be fastened to the neck either by way of ties at either end of the cuff or by using hook and loop fastening such as Velcro® on the ends of the cuff. Positioned at defined locations 11a, 11b are pressure engaging pressure applicators which can be positioned so that they come into contact with the carotid artery on either side of the neck of an individual as shown in FIG. 1. The pressure applicators in this case comprise balloon type members which can be inflated and deflated when in contact with the carotid arteries to cause occlusion or partial occlusion and release from the arteries. Also shown, are visual indicators 12 which a healthcare professional can use to align the pressure applicators with the carotid arteries of the neck of a person. In an alternative arrangement, or in combination with the visual indicators, there may be sensors 13 which can sense a pulse at the carotid arteries and provide a signal which may be either audible or visible or a combination of both so that when the pressure applicators are correctly positioned over the carotid arteries, the process of occluding the arteries can be started. Although balloon type pressure applicators are shown, these applicators could be for example mechanical feet which have actuators to bring them into contact with the carotid artery or alternatively, may be provided as protrusions which lie outside the plain of the neck engaging member 10 such that when the cuff is tightened, the pressure applicators press against the carotid artery.

The operation of the device is precisely monitored so that the application of pressure occurs at just the right time and for just the right length of time to provoke a cardiac event. A control mechanism 14 can be used to detect R-waves associated with cardiac activity and on detection of the R-wave can cause actuators to apply pressure to the carotid arteries via the pressure applicator. The control mechanism may itself control the pressure applied and the length of time applied or alternatively, it can be associated with a separate control with which it interacts to make sure that the pressure is applied in the correct way to provoke a HRT response. Typically, pressure on the neck is maintained at less than 40 millimetres of mercury because above this level, there may be discomfort to the individual.

The control mechanism 14 is in communication with a number of controllers or sensors for example there is a pressure sensor which can detect the carotid pulse when the pressure applicator is positioned over the carotid artery and this allows for precise alignment of the pressure applicators with the carotid arteries and also, the sensor can include means to monitor the pulse rate of the individual. For example if the pulse rate is particularly slow or weak, then the pressure applied by the occlusion or partial occlusion device can be altered so that less pressure is applied to the neck which reduces the possible damage to arteries. The sensor or an alternative sensor can also be used to detect the R-wave so that pressure can be applied once this has occurred. The R-wave can act as a trigger mechanism for an actuator which causes the pressure applicator to come into contact with the at least one carotid artery. Typically, the sensor is a solid state sensor but other pressure sensors could be used which the skilled person would understand would be applicable to the invention.

While occlusion or partial occlusion occurs to the arteries, pressure is measured for example through a pressure measuring device which measures the pressure of the pressure applicator against the carotid artery and this for example may be measured using a manometer. However, other pressure measuring devices could be used.

There is a control system in communication with the control mechanism which can receive pressure feedback from the pressure measuring device so that this can be compared with data such as a reference signal to ensure that the occlusion or partial occlusion of the carotid artery is within acceptable predetermined limits. If it is detected that the pressure is too great, the control mechanism can release the pressure of the pressure applicator. There is also an additional safety mechanism such that should the device fail, the application of pressure would be ceased immediately. Included as part of the safety feature are limiters for current value so that a reduction in current limits the activity of the actuator to reduce pressure applied to the carotid artery. A further desirable feature is the use of an ultrasound Doppler probe to monitor the state of the artery walls and detect should material be dislodged from the artery walls such that if this is detected, an alarm can be emitted and healthcare professionals would be alerted to the fact that they may need to provide clot reducing drugs to minimise the risk of damage to the individual.

FIG. 6 shows a variation of the device shown in FIG. 5 where arms 15 which are in contact with an actuator 16 come into contact with the neck at the carotid artery region. The arms 15 provided as mechanical arms that are hinged at point 17. A control equivalent to that as shown in FIG. 5 as 14 also controls the activity of the device. The device may be held by a healthcare professional or placed on a stand and the individual puts his or her neck between the arms and the arms are caused to move towards and abut against the neck of the individual. This is the first stage of operation. Following this controlled pressure can then be applied to the neck. The control mechanism controls delivery of pressure to the arms so that the arteries are occluded or partially occluded.

FIG. 7a shows a device which includes a neck cuff type arrangement for securing to the neck as shown in FIG. 5 but which includes a mechanical foot type arrangement for occluding or partially occluding the carotid arteries.

The device comprises a cuff 10 for encircling the neck and at either end of the cuff there are areas of hook and loop, otherwise known as Velcro® fasteners (10a, 10b). There is a pressure foot support unit 18, which has flanged slots 19, which can receive a pressure foot (shown in FIG. 7b).

As shown in FIG. 7b, the device shown in FIG. 7a from side on includes the fixed part 20 of a voice coil actuator. This is arranged in or on the cuff 10 to be remote from the part of the device i.e. the foot that comes into contact with the individual's neck.

The moving element 21 of the voice coil actuator can move towards and away from the pressure foot 22 that comes into contact with the or both carotid arteries. Associated with the pressure foot 22 is an ultrasound probe 23 which can detect events in the carotid arteries such as pulse or the loosening of plaque material as well as blood flow. There is also a pressure sensor 24 which can detect the pressure being applied to the individual's neck and which is associated with a controller so the pressure is only applied within certain defined parameters or limits.

Moving on to FIG. 8, this diagram represents a schematic arrangement of the safety features and controls that may be used with a device according to the invention. The controls can be used with the balloon type occlusion device of FIG. 5, the mechanical arm type device of FIG. 6 or the cuff and mechanical foot type arrangement as shown in FIGS. 7a and 7b.

The carotid arteries of the neck of an individual are shown as A. A neck cuff is placed around the neck (not shown) and pressure feet 22 sit in proximity to the carotid arteries.

The pressure feet 22 are caused to come into contact with the carotid arteries by activation of voice coil activators 20 which are operated by voice coil drivers 27. The voice coil drivers are controlled by controller 26. The controller also receives current feedback 25 as well as feedback from pressure sensor 24, which is associated with the mechanical feet. The activation of the voice coil actuators will be controlled by measurement of pressure level feedback 30. There is also a processor 3 which monitors heart rhythm. In addition an ECG monitor gives an indication of the heart rhythm. When R-wave is detected and R-wave trigger 28 causes pulse generation 29 by way of the controller 26.

One or both of the supports of the pressure foot 22, usually in the form of a sac in which the pressure foot is contained, will contain a pressure transducer that produces feedback to control the voice coil pressure actuator. Feedback will ensure that the stroke length of the actuator does not produce overpressure. Additionally there is an opportunity within the feedback loop to optimise the pressure impulse shape.

Independent voice coil actuators will compensate for asymmetric hydraulic transmission from neck to carotid sinus.

The pressure sensors in the sacs will also serve as a pulse detector to enable optimum positioning over the carotid sinuses.

Safe control of the pressure pulse amplitude will be managed through the feedback system and by monitoring the voice coil actuating current.

Analysis of the HRT sequences will be performed using the ECG data.

As can be seen, the arrangement of the invention has particular features to ensure safety of operation. Other features that ensure safety are those which minimise the risk of electric shock, for example the voice coil pressure driver is a low voltage device (12v-24v). Isolation of the control system with medical grade design meeting IEC 60601-1 requirements will minimise electric shock issues. Also it is preferred that the ECG recording circuit is isolated and is powered by batteries or medical grade power supplies.

Further carotid sinus pressure is avoided by the incorporation of a pressure sensor within the pressure transmission fluid and feedback controlling signal to actuator driver. Also it is possible to incorporate an additional driver current monitoring circuit or actuator limit detectors or actuator current damping.

Further, the device is preferably biocompatible with individuals and is made of medical grade material to reduce the risk of allergic reaction.

Finally, patient trauma is minimised by selecting patients with ultrasound pre-scan to evaluate the suitability of patients with carotid plaque. Also there is heart rate monitoring derived from sac pressure sensor and ECG sensor.

The invention covers not only individual embodiments as discussed but combinations of embodiments as well. It is to be understood that modifications and variations of the present invention will become apparent to those skilled in the art and it is intended that all such modifications will be included within the scope of the present invention.

1. A device for altering cardiac activity, said device comprising a neck engaging member, said neck engaging member having at least one pressure applicator provided as a predefined area which in use comes into contact with and occludes or partially occludes at least one carotid artery, said device including a control mechanism which is operable to cause the pressure applicator to rapidly occlude or partially occlude the at least one carotid artery in order to provoke heart rate turbulence.

2. A device according to claim 1, wherein the device includes a control mechanism that causes the pressure applicator to occlude or partially occlude the at least one carotid artery and release therefore after a predetermined period of time.

3. A device according to claim 1, wherein the occlusion or partial occlusion of the at least one carotid artery is achieved within one or more milliseconds.

4. A device according to claim 1, wherein occlusion or partial occlusion of the at least one carotid artery is achieved within 5 to 20 milliseconds.

5. A device according to claim 3, wherein the occlusion or partial occlusion has a duration for one or more cardiac cycles.

6. A device according to claim 1, wherein the neck engaging member device comprises a cuff that is placed around the neck of the individual, with the pressure applicator being aligned with the at least one carotid artery.

7. A device according to claim 6, wherein the pressure applicator is an inflatable balloon that when inflated occludes or partially occludes the at least one carotid artery.

8. A device according to claim 6, wherein the pressure applicator is a mechanical foot that comes into contact with and occludes or partially occludes the at least one carotid artery.

9. A device according to claim 1, wherein the neck engaging member comprises one or more mechanical arms that come into contact with the one or more carotid arteries.

10. A device according to claim 1, wherein the neck engaging member includes a pressure sensor, which detects the carotid pulse when the pressure applicator is positioned over the carotid artery.

11. A device according to claim 1, wherein the neck engaging member includes visible indicators so that the neck engaging member can be aligned with the carotid artery.

12. A device according to claim 1, wherein the pressure applicator is a mechanical foot that is brought into contact with the at least one carotid artery.

13. A device according to claim 1 including a cardiac monitoring sensor to detect the R-wave associated with cardiac activity.

14. A device according to claim 13, wherein the cardiac monitoring sensor is in communication with an actuator that is operable to cause the pressure applicator to come into contact with the at least one carotid artery following detection of the R-wave.

15. A device according to claim 14, wherein activation of the actuator is triggered by detection of the R-wave followed by which the actuator is caused to release so that zero pressure is applied to the at least one carotid artery.

16. A device according to claim 14, wherein the actuator is a voice coil actuator.

17. A device according to claim 1, wherein the device includes a pressure measuring device that monitors the pressure of the pressure applicator against the carotid artery.

18. A device according to claim 17, wherein the pressure measuring device is a manometer.

19. A device according to claim 1, including a control system that can receive a pressure feedback signal from the pressure measuring device so that said signal can be compared with a reference signal.

20. A device according to claim 1, wherein the reference signal is stored as a value within predetermined safety levels for pressure that can be applied to the carotid artery.

21. A device according to claim 1, including a limited for the current to the actuator so that the pressure applied to the carotid artery is limited by reduction of the current.

22. A device according to claim 1 including an ultrasound doppler probe.

23. A device according to claim 1 including a timer device.

24. A device according to claim 1 arranged to occlude or partially occlude more than one carotid artery.

25. A device according to claim 1 to alter the heart rate of animals or humans.

26. A method of occluding the carotid artery, said method comprising applying a neck engaging member to an individual's neck such that at least one pressure applicator provided as a predefined area comes into contact with at least one carotid artery, operating a control mechanism to cause the pressure applicator to rapidly occlude or partially occlude the at least one carotid artery in order to provoke heart rate turbulence.

27. A method according to claim 26, wherein the control mechanism is caused to occlude or partially occlude the at least one carotid artery and release therefore after a predetermined period of time.

28. A method according to claim 23, wherein the device is caused to occlude or partially occlude the at least one carotid artery within between 5 and 20 milliseconds for a duration of one or more cardiac cycles.

29. (canceled)

30. (canceled)
